(19) United States
(12) Patent Application Publication
(10) Pub. No.: US 2026/0124009 A1
(43) Pub. Date: May 7, 2026

(54) Robotic Dental System and Method of Preparing for a Robotic Dental Procedure

(71) Applicant: Perceptive Technologies, Inc., Boston, MA (US)

(72) Inventors: Justin LaRue, Cambridge, MA (US); Christopher John Ciriello, Boston, MA (US); Phillip Getto, Wellesley, MA (US); Scott Kilcoyne, Cambridge, MA (US); Alexander Krull, Wellesley, MA (US); Jesse Mitchell, Grafton, MA (US); Joseph Doeringer, Swampscott, MA (US); Haoran Wang, Boston, MA (US); Jack Mondry, Medford, MA (US)

(21) Appl. No.: 19/383,543

(22) Filed: Nov. 7, 2025

Related U.S. Application Data
(60) Provisional application No. 63/717,862, filed on Nov. 7, 2024.

(51) Int. Cl. A61B 34/32; A61B 90/00; A61C 3/10; A61G 15/14

(52) U.S. Cl. CPC A61B 34/32; A61C 3/10; A61G 15/14; A61B 2090/064

(57) ABSTRACT

A dental robot includes a platform and a suspension system. The suspension system supports the platform and automatically compensates for weights of the platform and a robot arm, end effector, and tooth clamp attached to the platform. The suspension system maintains a constant height of the platform above a base, absent an external force on the tooth clamp. The suspension system allows position and orientation of the platform to change with three degrees of positional freedom and three degrees of rotational freedom, relative to the base, in response to an external force on the tooth clamp, such as a result of movement (change in position and/or orientation) of a subject. A rigid connection between the tooth clamp and the platform ensures that position and orientation of the platform remains fixed, relative to the subject's teeth, in response to movement of the teeth.

### CROSS REFERENCE TO RELATED APPLICATIONS

This application claims a benefit of U.S. Provisional Ser. No. 63/717,862, filed Nov. 7, 2024, titled “Robotic Dental System and Method of Preparing for a Robotic Dental Procedure,” the entire contents of which are hereby incorporated by reference herein, for all purposes.

### TECHNICAL FIELD

The present disclosure relates to dental robotics.

### RELATED ART

The access to dental care crisis in the United States warrants immediate attention. Over 90% of American adults are affected by dental caries, and approximately 35% of Americans do not visit a dentist annually, with 28% having untreated tooth decay. This widespread dental care avoidance is often attributed to high costs and long appointments. This avoidance contributes to over $45 billion in lost productivity and over 34 M lost school hours for young adults and has severe ramifications for individuals' overall health, including increased risks of diabetes, cardiovascular disease, and Alzheimer's disease.

The current state of dentistry has numerous challenges, including a heavy reliance on manual procedures that incur high costs and a limited supply of dental practitioners.

To address these problems we propose the development of a highly precise and accurate robotic tooth preparation system.

The following references may be relevant to the present disclosure: U.S. Pat. Publ. No. 2016/0367343 to Mozes, et al., U.S. Pat. Publ. No. 2016/0354169 to Suttin, and WO 2017/100828 to Zuaiter, et al.

Reference is also made to commonly-owned PCT application, pub. no. WO2024/229470A1, the entire contents of which are hereby incorporated by reference herein, for all purposes.

### SUMMARY OF EMBODIMENTS

The present disclosure relates to dental robotics, and more particularly to a dental robot that can automatically compensate for weight of a robot arm and can automatically maintain a constant position and orientation of a proximal end of the robot arm, relative to teeth of a human subject, throughout the course of a dental surgery procedure.

In a first aspect, the present disclosure provides a robotic dental system, comprising: a treatment system, a base, and a suspension system.

The treatment system comprises: a robotic arm, a distal end of which is configured to be coupled to an end effector; and a platform, to which a proximal end of the robotic arm is coupled, the platform comprising a coupling portion for rigidly coupling to a dental clamp, which is configured to be rigidly clamped to one or more teeth of a subject, the platform and coupling portion being configured such that, when the dental clamp is rigidly clamped to the one or more teeth and the dental clamp is rigidly coupled to the coupling portion, position and orientation of the platform remain fixed, relative to the one or more teeth.

The suspension system comprises a proximal end, which is coupled to the base, and a distal end, which is coupled to the platform. The suspension system supports a weight of the treatment system, and is configured such that, when the robotic dental system is operating in a treatment mode, with the dental clamp rigidly clamped to the one or more teeth and the dental clamp rigidly coupled to the coupling portion, the suspension system permits a the position and orientation of the platform to change, relative to the base, in response to forces applied by the one or more teeth to the dental clamp, thereby accommodating changes in the position, orientation, and both of the one or more teeth by enabling corresponding changes in the position, orientation, or both of the platform.

The suspension system comprises a plurality of linkages, connected by a plurality of joints, the plurality of linkages and the plurality of joints together providing the platform with three degrees of translational freedom and three degrees of rotational freedom.

In some examples, the plurality of joints comprises a plurality of vertical axis revolute joints, each of which is connected to one or more of the plurality of linkages, and enables said one or more linkages to rotate about a vertical axis corresponding to the revolute joint in question, and

wherein the plurality of vertical axis revolute joints provides the platform with two degrees of translational freedom in a horizontal plane. The plurality of vertical axis revolute joints can, for example, comprise three vertical axis revolute joints. In specific examples, the plurality of vertical axis revolute joints additionally provides the platform with a rotational degree of freedom about a vertical axis.

In some examples, a proximal-most of the plurality of vertical axis revolute joints is located at the proximal end of the suspension system. In addition, or instead, one or more of the plurality of vertical axis revolute joints can, for example, be coupled to respective motors, each of which is configured to cause movement of the corresponding vertical axis revolute joint.

In some examples, the plurality of joints comprise one or more elevation joints, which provide the platform at least with a translational degree of freedom in a vertical direction. In specific examples, at least a first elevation joint of the one or more elevation joints is coupled to at least one motor, which is configured to cause movement of the first elevation joint. In more specific examples, the system further comprises one or more force sensors, which are operable to sense force applied by the one or more teeth to the dental clamp, wherein the at least one motor coupled to the first elevation joint is controlled based on an output from the one or more force sensors. Additionally, or alternatively, the first elevation joint is coupled to at least one mechanical force-generating element, which applies force that counteracts force applied to the elevation joint by a portion of the suspension system distal to the elevation joint.

In some examples, the one or more elevation joints are revolute joints. In specific examples, where at least a first elevation joint of the one or more elevation joints is coupled to at least one motor, which is configured to cause movement of the first elevation joint, a maximum torque applied to the elevation joint by the at least one mechanical force-generating element can, for instance, be greater than a maximum torque applied to the elevation joint by the at least one motor. In more specific examples, the maximum torque applied to the elevation joint by the at least one mechanical force-generating element is at least 2 times greater, at least 4 times greater, at least 6 times greater, or at least 8 times greater than the maximum torque applied to the elevation joint by the at least one motor.

In some examples, the elevation joint is a prismatic joint. In specific examples where at least a first elevation joint of the one or more elevation joints is coupled to at least one motor, which is configured to cause movement of the first elevation joint, a maximum force applied to the elevation joint by the at least one mechanical force-generating element can, for instance, be greater than a maximum force applied to the elevation joint by the at least one motor. In more specific examples, the maximum force applied to the elevation joint by the at least one mechanical force-generating element is at least 2 times greater, at least 4 times greater, at least 6 times greater, or at least 8 times greater than the maximum force applied to the elevation joint by the at least one motor.

In some examples, the plurality of joints comprises one or more non-vertical axis rotational joints, which collectively provide the platform with two rotational degrees of freedom about respective, non-vertical axes. In examples where the plurality of joints comprise one or more elevation joints, which provide the platform at least with a translational degree of freedom in a vertical direction, the one or more non-vertical axis rotational joints can, for example, be located distally of the elevation joint.

In some examples, the one or more non-vertical axis rotational joints can be located distally of the plurality of vertical axis revolute joints.

In some examples, the one or more non-vertical axis rotational joints can, for example, be coupled to one or more motors, which are configured to cause movement of the one or more non-vertical axis rotational joints. In specific examples, the system further comprises one or more force sensors, which are operable to sense force applied by the one or more teeth to the dental clamp, wherein the one or more motors coupled to the one or more non-vertical axis rotational joints are controlled based on an output from the one or more force sensors. Additionally, or alternatively, the one or more non-vertical axis rotational joints can, for example, be coupled to one or more mechanical force-generating elements, which apply torque that counteract torque applied to the one or more non-vertical axis rotational joints by a portion of the suspension system distal to the one or more non-vertical axis rotational joints. In specific examples, a maximum torque applied to the one or more non-vertical axis rotational joints by the one or more mechanical force-generating elements is greater than a maximum torque applied to the elevation joint by the one or more motors. In still more specific examples, the maximum torque applied to the one or more non-vertical axis rotational joints by the one or more mechanical force-generating elements is at least 2 times greater, at least 4 times greater, at least 6 times greater, or at least 8 times greater than the maximum torque applied to the one or more non-vertical axis rotational joints by the one or more motors.

In some examples, the plurality of joints comprises one or more active joints, each of which is driven by at least one motor, and wherein the one or more active joints are configured so as to apply forces of less than 40 N, less than 20 N, less than 10 N, or less than 5 N to the one or more teeth of the subject through the dental clamp. In specific examples, each of the one or more active joints is configured so as to apply forces of less than 40 N, less than 20 N, less than 10 N, or less than 5 N to the one or more teeth of the subject through the dental clamp.

In some examples, the robotic dental system is configured to cause the robotic arm to perform a dental procedure on at least one target tooth of the one or more teeth to which the dental clamp is rigidly clamped. The robotic dental system disclosed herein may achieve particularly high accuracy when carrying out a dental procedure on one or more of the teeth clamped by the dental clamp.

In some examples, the plurality of joints comprise a plurality of passive joints that provide the platform with two degrees of translational freedom in a horizontal plane. In such examples, the plurality of passive joints can, for example, comprise a plurality of passive revolute joints, each of which is connected to two or more of the plurality of linkages, and enables said two or more linkages to rotate relative to one another about a vertical axis corresponding to the revolute joint in question. Additionally, or alternatively, the plurality of joints can, for example, comprise: at least one passive joint that provides the platform with a rotational degree of freedom about a vertical axis; one or more active joints that provide two rotational degrees of freedom about respective axes that are angled with respect to the vertical axis. Additionally, or alternatively, the plurality of joints can, for example, comprise one or more passive elevation joints, which provide the platform with at least a translational degree of freedom in a vertical direction. In such examples, the one or more passive elevation joints can, for example, comprise one or more passive rotational elevation joints, each of which is connected to two or more the plurality of linkages, and enables the two or more linkages to rotate relative to one another about a horizontal axis corresponding to the passive rotational elevation joint in question. In such examples, the two or more linkages connected to each passive rotational elevation joint can, for example, comprise a lever linkage, said lever linkage comprising opposing first and second portions, the passive rotational elevational joint being connected to the lever linkage between said first and second portions, wherein a weight of the treatment system is supported by the respective first portions of the lever linkages connected to the one or more passive rotational elevation joints, and wherein a counterbalancing weight is mounted on the respective second portions of the lever linkages connected to the one or more passive rotational elevation joints, and the counterbalancing weight substantially counterbalances the weight of the treatment system.

In examples where the plurality of passive joints comprises a plurality of passive revolute joints, each of which is connected to two or more of the plurality of linkages, and enables said two or more linkages to rotate relative to one another about a vertical axis corresponding to the revolute joint in question, the plurality of passive revolute joints can, for example, be disposed proximally of the one or more passive elevation joints.

In some examples, the plurality of joints comprise: one or more active rotational elevation joints, which provide the platform with at least a translational degree of freedom in a vertical direction, each active rotational joint being connected to two or more the plurality of linkages, and enabling the two or more linkages to rotate relative to one another about a horizontal axis corresponding to the active rotational elevation joint in question. In such examples, the two or more linkages connected to each active rotational elevation joint can, for example, comprise a lever linkage, said lever linkage comprising opposing first and second portions, the active rotational elevational joint being connected to the lever linkage between said first and second portions, wherein a weight of the treatment system is supported by the respective first portions of the lever linkages connected to the one or more active rotational elevation joints, and wherein a counterbalancing weight is mounted on the respective second portions of the lever linkages connected to the one or more passive rotational elevation joints, and the counterbalancing weight substantially counterbalances the weight of the treatment system.

In examples where the plurality of passive joints comprises a plurality of passive revolute joints, each of which is connected to two or more of the plurality of linkages, and enables said two or more linkages to rotate relative to one another about a vertical axis corresponding to the revolute joint in question, the first and second passive revolute joints can, for example, be disposed proximally of the one or more active elevation joints, or the active rotational elevational joints (as the case may be).

Optionally, in any embodiment, the system may further comprise a leveling system, which comprises one or more moveable elements that are operable to maintain the suspension system in a predetermined orientation with respect to a vertical direction. In some examples, the base comprises the leveling system.

Optionally, in any embodiment, the suspension system comprises: a gross positioning system, which comprises: a plurality of gross positioning linkages, which are comprised by the plurality of linkages of the suspension system, and a plurality of gross positioning joints, which connect the plurality of gross positioning linkages and are comprised by the plurality of joints of the suspension system; an admittance system, which comprises: a plurality of admittance linkages, which are comprised by the plurality of linkages of the suspension system, and a plurality of admittance joints, which connect the plurality of admittance linkages and are comprised by the plurality of joints of the suspension system. The gross positioning system connects, at least in part, the admittance system to the base, and, when the robotic dental system is operating in a setup mode, the gross positioning system enables a user of the robotic dental system to move the admittance system into a desired arrangement relative to the subject. The gross positioning joints are configured to be locked, so as to prevent relative movement of the gross positioning linkages during the treatment mode. When the robotic dental system is operating in a treatment mode, the admittance system permits the position and orientation of the platform to change, relative to the base, in response to forces applied by the one or more teeth to the dental clamp, thereby accommodating changes in the position, orientation, and both of the one or more teeth by enabling corresponding changes in the position, orientation, or both of the platform.

Optionally, in any embodiment, the treatment system comprises at least one force sensor, and wherein, when the robotic dental system is operating in a compliant mode, with the dental clamp rigidly clamped to the one or more teeth and the dental clamp rigidly coupled to the coupling portion: the suspension system permits the position and orientation of the platform to change, relative to the base, in response to forces applied by the one or more teeth to the dental clamp, thereby accommodating changes in the position, orientation, and both of the one or more teeth by enabling corresponding changes in the position, orientation, or both of the platform, and the treatment system; and the robotic arm is operated, based on input from the at least one force sensor of the treatment system, in response to forces applied to the treatment system by an operator of the robotic dental system, thereby permitting the operator to reposition the robotic arm in a desired arrangement.

Optionally, in any embodiment with an active suspension system, when the robotic dental system is operating in a treatment mode, the at least one motor is operated based on input from at least one force sensor so as to cause the position, orientation or both of the platform to change, relative to the base, in response to the forces applied to the dental clamp, thereby permitting corresponding changes in the position, orientation, or both of the one or more teeth.

Optionally, in any embodiment with an active suspension system, when the robotic dental system is operating in a compliant mode, the at least one motor is operated, based on input from at least one force sensor, so as to cause the position, orientation or both of the platform to change, relative to the base, in response to forces applied to the treatment system by an operator of the robotic dental system, thereby permitting the operator to reposition the treatment system in a desired arrangement.

Optionally, any embodiment further includes the clamp, rigidly coupled to the coupling portion.

Optionally, in any embodiment that includes the clamp, the dental clamp is configured to be rigidly clamped to a plurality of teeth of the subject, and the robotic arm is operable to address at least two of the plurality of teeth.

Optionally, in any embodiment, the robotic arm is configured so as to be insertable into a mouth of the subject separately from the dental clamp.

Optionally, in any embodiment, the robotic dental system is configured to cause the robotic arm to perform a dental procedure on at least one target tooth of the one or more teeth to which the dental clamp is rigidly clamped.

### BRIEF DESCRIPTION OF THE DRAWINGS

The present disclosure will be more fully understood by referring to the following Detailed Description of Specific Embodiments in conjunction with the Drawings, in which:

FIG. 1 is a schematic side view of an example of a robotic dental system that demonstrates design principles incorporated in robotic dental systems according to the first aspect of this disclosure.

FIGS. 2 and 3 illustrate the robotic dental system of FIG. 1 with a subject in two different positions and orientations.

FIG. 4 is schematic side view of the robotic dental system of FIG. 1, when implemented with an active suspension system.

FIGS. 5 and 6 are schematic side views of a comparative example of a robotic treatment system, with a subject in different positions and orientations.

FIG. 7 is a perspective view of a miniature robotic dental treatment system, according to a comparative example.

FIG. 8 is a schematic side view of a robotic dental system according to a first aspect of this disclosure.

FIGS. 9 and 10 are schematic top views of the robotic dental system of FIG. 8 illustrating respective points in time during movement of the platform as a result of articulation of vertical axis revolute joints.

FIG. 11 is a schematic side view of an example of a robotic dental system in which a spring is coupled to an active elevation join.

FIG. 12 is a schematic side view of an example of a robotic dental system in which a counterweight is coupled to an active elevation joint.

FIG. 13 is a schematic side view of an example of a robotic dental system having a prismatic elevation joint.

FIG. 14 is a schematic side view of a further example of a robotic dental system whose suspension system includes both active and passive joints.

FIG. 15 is a schematic side view of a still further example of a robotic dental system, which includes a counterbalancing arrangement.

FIG. 16 is a side view of a further example of a suspension system with a counterbalancing arrangement.

### DETAILED DESCRIPTION OF SPECIFIC EMBODIMENTS

An automated robotic dental treatment system described herein may be capable of achieving an accuracy of at least about 50 microns (μm) in automating tooth preparation for dental crowns and other dental procedures. This accuracy is an order of magnitude more accurate than current dental robotic systems, surpassing the performance of existing surgical robots, such as Yomi (NeoCis, Inc., Miami, FL, USA) and THETA (Hangzhou Jianjia Robot Co., Ltd., Hangzhou, China), which only have an accuracy of about 750-1100 μm.

In order to achieve high accuracy, two conventional approaches have been to, one, dynamically register a robot or, two, make the robot so small and light weight that it can completely fit onto a target tooth.

A robot is an automated machine capable of executing a specific task with minimal human intervention (i.e., autonomously) while maintaining speed and precision. A serial manipulator is a type of robot that includes a series of links connected by motor-actuated joints that extend from a base to an end effector. The motor-actuated joints may include but are not limited to linear joints, rotational joints, and spherical joints, and such joints may be provided with sensors for one or more of position, orientation, or force, such as linear transducers, haptic sensors, torque sensors, accelerometers, gyroscopes, and magnetic or visual indicators for external sensors.

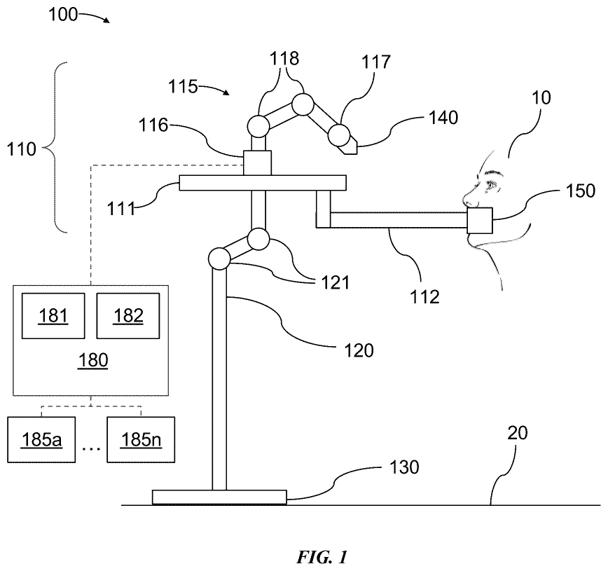

Reference is now direction to FIGS. 1-3, which are schematic side views of a robotic dental system 1000 that embodies certain design principles that are implemented within robotic systems according to aspects of this disclosure. FIGS. 1-3 show a subject 10 (e.g. a patient) in different positions and orientations, during treatment with the robotic dental system 1000.

As may be seen from FIG. 1, the robotic dental system 1000 comprises a robotic arm 115, and a platform 111, to which a proximal end 116 of the robotic arm 115 (which may, in some examples, be referred to as the root, shoulder, or base of the robotic arm 115) is attached. A distal end 117 of the robotic arm 115 is coupled to an end effector 140, which is used for carrying out a dental procedure on subject 10. Accordingly, the robotic arm 115 may, in examples, be referred to as a “treatment arm” and the end effector 140 can, for example, be a dental drill. The distal end 117 of the robotic arm 115 can comprise a suitable receptacle for coupling to the end effector 140. The receptacle can, for example, comprise a mechanical interface to ensure the end effector is coupled to the distal end 117 with the right orientation. Examples of end effectors suitable for use with the robotic treatment systems of this disclosure are described in U.S. patent applications Ser. No. 17/054,442, filed Jun. 22, 2021, Ser. No. 17/054,445 filed Nov. 10, 2020, and Ser. No. 18/066,892, filed Dec. 15, 2022.

As indicated in FIG. 1, the platform 111 and the robotic arm 115 are part of a treatment system 110 of the robotic dental system 1000. As will be explained in more detail below, the treatment system 110 is able (with the aid of a suspension system 120 of the robotic dental system 1000) to accommodate movement by the subject 10 during treatment, thereby promoting the comfort of the subject 10 during treatment, while maintaining the accuracy of the robotic arm 115.

As shown in FIG. 1, the platform 111 comprises a coupling portion 112, which is rigidly (and removably/releasably) coupled to a dental clamp 150. By “rigidly coupled”, it is meant that such coupling maintains the coupling portion 112 in a fixed position and orientation relative to the dental clamp 150, even if the subject 10, and therefore the dental clamp 150, changes position or orientation. As a result of the rigid coupling, the platform 111 likewise remains in a fixed position and orientation relative to the dental clamp 150. Although in the example shown in FIGS. 1-3 the coupling portion 112 is shown as being in the form of an arm extending from the platform 111, this is by no means essential and the coupling portion 112 can have any suitable structure to allow rigid coupling to a particular design of dental clamp 150. For example, the coupling portion 112 and the dental clamp 150 can comprise any suitable coupling features that permit one to rigidly couple with the other such as a pin, a threaded feature, a clip, a magnet, a nut, a bolt, a catch, a clasp, or any combination thereof.

The platform 111 functions to maintain various fixtures that are attached thereto in a fixed position and orientation relative to each other. Accordingly, the platform 111 can, for example, be a substantially rigid structure. While in the example of a robotic dental system 1000 shown in FIG. 1, the fixtures attached to the platform 111 include the dental clamp 150 (by way of the coupling portion 112 of the platform 111) and the robotic arm 115, it will be appreciated that, in other examples, the platform 111 may be configured so that additional fixtures may be rigidly coupled thereto, such as further robotic arms, or mechanical arms that can be coupled to intraoral scanning devices (e.g., as described in WO2022/212507A1). Furthermore, while in the example of a robotic dental system 1000 shown in FIG. 1, the platform 111 is configured with a generally planar main body, this is by no means essential and in other examples the platform 111 may have any suitable shape that permits it to maintain fixtures coupled thereto in a fixed position and orientation.

Though not shown in detail in FIG. 1, dental clamp 150 is in turn rigidly (and removably) coupled to one or more teeth of the subject 10. Examples of suitable clamps for use with the robotic dental systems described herein are described in U.S. patent application Ser. No. 17/054,442, filed Jun. 22, 2021, Ser. No. 17/054,445 filed Nov. 10, 2020, and Ser. No. 18/066,892, filed Dec. 15, 2022. When clamping to the one or more teeth of the subject 10, the dental clamp 150 directly contacts and clamps onto the teeth themselves. As a result, the position and orientation of the platform 111 remain fixed, relative to the one or more teeth clamped by the dental clamp 150 (even if the subject 10, and therefore the dental clamp 150, changes position or orientation). Such an arrangement may assist the robotic arm 115 in accurately addressing the teeth of subject 10, particularly (but not exclusively) where the robotic dental system 1000 is configured (e.g., by suitable programming of the processor(s) 181 of control system 180) to perform a procedure on one or more of the teeth that are clamped by the dental clamp 150.

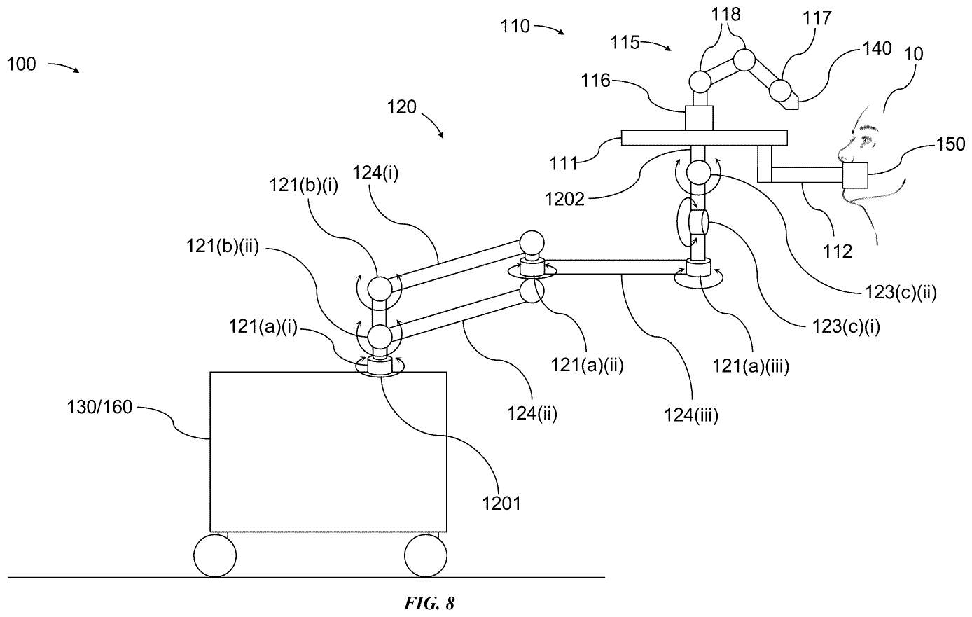

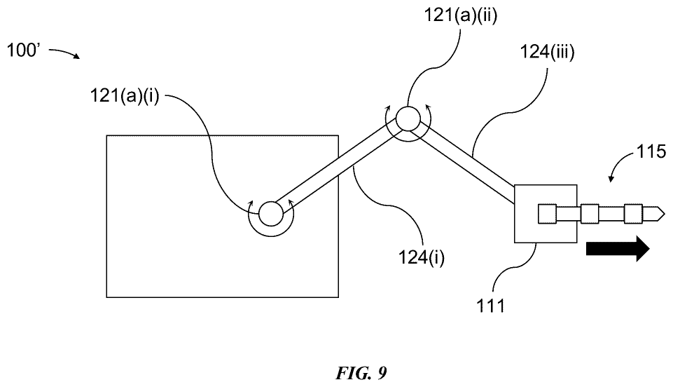

As be seen from FIG. 1, the robotic dental system 1000 further comprises a base 130, and a suspension system 120, which mechanically couples the platform 111 with the base 130. The base 130 of the robotic dental system 1000 remains generally stationary during a procedure and hence remains in a generally fixed position and orientation with respect to the environment in which the robotic dental system 1000 is operating (e.g., a room or space in a dental practice where the procedure is being carried out). In the example shown in FIG. 1, the base 130 is fixed to the floor 20 (or to the ground). However, this is by no means essential and in other examples, the base 130 may simply rest on the floor 20 (or the ground), for instance on wheels (e.g., having casters) provided on the base 130. In still other examples, such as that shown in FIGS. 8-9, the base 130 may be fixed to a moveable module that supports the treatment system 110 and the suspension system 120 and, optionally, provides other components of the robotic dental system 100). Still further, it should be appreciated that the base 130 could be fixed to a wall or ceiling of the room or space in which the procedure is being performed, rather than the floor 20.

As further shown in FIG. 1, the robotic dental system 1000 may additionally comprise a control system 180 that comprises at least one processor 181. The control system 180 governs the operation of various subsystems within the robotic dental system 1000 (e.g., as a result of suitable programming of the at least one processor 181). In particular, as shown by the dashed line in FIG. 1, the control system 180 may be in data communication with the robotic arm 115 so as to control the movement thereof. As shown in FIG. 1, the control system 180 additionally comprises computer-readable storage medium 182, which stores instructions for execution by the at least one processor 181 so that the robotic dental system 1000 operates as described herein.

In some examples, the control system 180 may be integrated into (and therefore form a part of) the robotic dental system 1000. However, in other examples, the robotic dental system 1000 may be configured such that it can be provided to an end-user without an integrated control system 180. In such cases the end user might, for example, use their own general purpose computer (such as a laptop) as a control system 180 for the robotic dental system 1000, for instance after downloading and installing suitable software on the general purpose computer.

Returning to FIG. 1, it may be noted that the control system 180 is additionally in data communication with one or more user input devices 185a-185n that, for example, enable an operator of the robotic dental system 1000 to provide instructions to the control system 180 that are followed by the robotic dental system 1000, for instance by the robotic arm 115 (or other subsystems of the robotic dental system 1000). As will be appreciated, various kinds of user input devices 185a-185n can be utilized, such as keyboards, joysticks, pointing devices (e.g., a computer mouse or trackball), touchscreen displays etc.

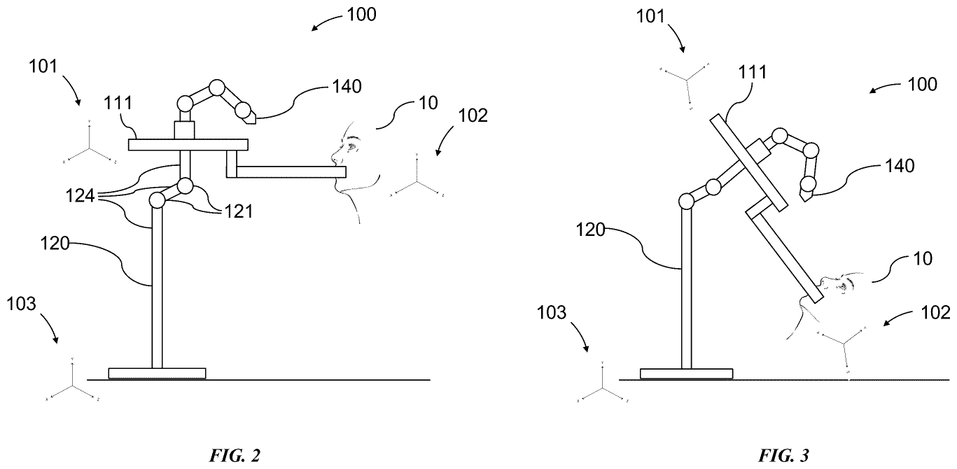

Reference is now directed to FIGS. 2 and 3, which are schematic side views of the robotic dental system 1000 of FIG. 1 that show a subject 10 (e.g. a patient) in respective different positions and orientations, and thereby enable the function of the suspension system 120 to be more fully understood.

FIG. 2 shows the subject 10 in a first position and orientation within a room or other environment where a dental procedure is being performed by robotic dental system 1000. Accordingly, FIGS. 2 and 3 both show the robotic dental system 1000 while it is operating in a “treatment mode”. During such a treatment mode the robotic dental system 1000 may, for example, operate substantially autonomously (e.g., receiving, at most, limited and/or high-level input from the operator). In particular, the robotic arm 115 may operate substantially autonomously to carry out a dental procedure on one or more target teeth within the mouth of the subject 10 (which, as noted above, may be one or more of the teeth clamped by the dental clamp 150). Such a treatment mode can be distinguished from, for example, a setup mode (e.g., where the robotic dental system 1000 is being moved into position). As shown in both FIGS. 2 and 3, the dental clamp 150 is clamped onto one or more teeth of the subject 10 and therefore the position and orientation of the platform 111 remain fixed, relative to the one or more teeth clamped by the dental clamp 150.

FIG. 3 shows the subject 10 a short time later, when he/she is in a second, different position and orientation within the environment. As may be appreciated from a comparison of FIGS. 2 and 3, this change in position and orientation of the subject 10 is accommodated by a corresponding change in the position and orientation of the platform 111. As is also apparent from a comparison of FIGS. 2 and 3, the platform 111 is able to change its position and orientation as a result of the action of the suspension system 120, specifically, by articulations of joints 121 in the suspension system 120. More particularly, the linkages 124 and joints 121 together provide the platform 111 with three degrees of translational freedom (i.e., movement in x, y and z directions) and three degrees of rotational freedom (e.g., pitch, yaw, and roll).

FIGS. 2 and 3 additionally show a frame of reference 101 for the platform 111 and a frame of reference 102 for the subject 10. As is apparent from FIGS. 2 and 3, the spatial relationship between these frames of reference 101, 102 is maintained when the subject 10 moves. Because the robotic arm 115 is rigidly coupled to the platform 111, whose frame of reference 101 is in a fixed spatial relationship with the frame of reference 102 for the subject 10, the robotic arm 115 can maintain its registration relative to the teeth of the subject 10, even when the subject 10 moves during a procedure.

While in the particular example shown in FIGS. 1-3, the suspension system 120 comprises a series of linkages 124 connected by joints 121, it will be appreciated that this particular structure is by no means essential and in other examples the suspension system may have any suitable structure that permits the position and orientation of the platform 111 to change, relative to the base 130, in response to forces applied by clamped tooth or teeth to the dental clamp 150, thereby accommodating changes in the position and orientation of the clamped tooth or teeth by enabling corresponding changes in the position and orientation of the platform 111. For example, suitable structures might comprise spring-loaded linkages, gimbals, linkages connected by friction joints and the like. Moreover, the suspension system 120 is not limited to a linear structure. Accordingly, in other examples, the suspension system could, for instance, include linkages in more complex arrangements, such as, for example, a four-bar linkage arrangement.

It should be appreciated that the suspension system 104 supports the weight of the treatment system 110, including the platform 111 and the robotic arm 115, so that they feel nearly weightless to the subject 10. Consequently, the platform 111 can “float” with respect to the floor 20 (or the ground). Hence (or otherwise), a relatively large and/or complex robotic arm 115 may be utilized in the robotic dental system 1000. For instance, the robotic arm 115 may have sufficient reach to be able to operate on several different teeth within the mouth of the subject 10, without needing the system to be reconfigured. In addition, or instead, the robotic arm 115 may, for example, be configured to permit movement of the distal end 117 in six degrees-of-freedom (DOF) with rotation (e.g., roll, pitch, yaw) and 3D translation of the end effector 140 with respect to the proximal end 116 of the robotic arm 115. Moreover, in some examples the robotic arm 115 may have more than six degrees-of-freedom, providing it with redundant degrees of freedom that can be used to provide improved access to the subject's mouth and/or the tooth/teeth to be treated (e.g., by enabling the robotic arm 115 to adopt an arrangement that is wider in the horizontal plane than the vertical plane, so as to better fit between the upper and lower dental arches), and/or to assist the operator in using the robotic dental system 1000 (e.g., by enabling the robotic arm 115 to adopt an arrangement that improves the operator's visibility of the tooth or teeth being treated by the robotic dental system 1000).

It should also be appreciated that the weights of the suspension system 120, the treatment system 110 (including the robotic arm 115 and the platform 111) may, in some examples, be selected so that the center of gravity of the full assembly of such components is above the base 130, for instance, even when the distal end 117 of the robotic arm 115 and/or the dental clamp 150 are fully extended away from the platform 111.

It should further be appreciated that the suspension system 120 may also (or instead) have redundant degrees of freedom (for instance, more than 6 degrees of freedom), for example allowing for its elbow to be moved into configurations that are convenient for an operator or subject.

Still further, it should be noted that the suspension system 120 may be configured either as a passive system or an active system. In a passive system, the accommodating movements of the suspension system 120 are not caused by powered components, but rather the suspension system 120 permits external forces that are applied to the treatment system 110 to mechanically cause the position and orientation of the platform 111 to change. For example, when subject 10 applies a force to the dental clamp 150 by attempting to move, that force is mechanically transmitted through the coupling portion 112, to the platform 111 and then to the suspension system 120, which moves, thus permitting the platform 111 to move, thereby enabling the subject 10 to move. In addition, where the suspension system 120 is configured as a passive system, an operator may be able to manually reposition the treatment system 110 during setup, for example by pushing and/or pulling the treatment system 110 so that it moves into a desired position and orientation relative to the subject 10.

Where, by contrast, the suspension system 120 is an active system, the accommodating movements of the suspension system 120 are caused by one or motors that form part of the suspension system 120. Such motors may, for example, comprise linear motors that cause linkages to translate relative to one another, and/or may comprise rotational motors that cause linkages to rotate relative to one another. In some examples, the suspension system 120 may essentially be a robotic arm or manipulator, whose movement is based on the output of force sensors.

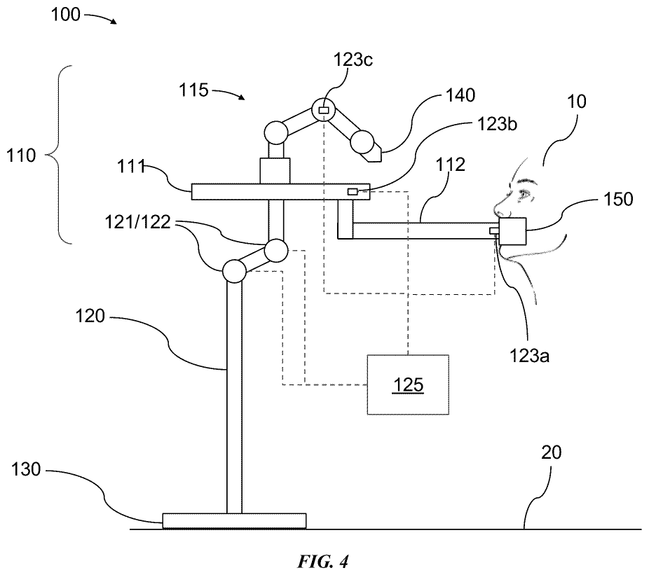

suspension system 120 An example of a robotic dental system 1000 with an active suspension system 120 is shown in FIG. 4. As is apparent, the robotic dental system 1000′ of FIG. 4 is a modified version of the robotic dental system 1000 of FIGS. 1-3. As is apparent, the robotic dental system 1000′ of FIG. 4 differs from that of FIGS. 1-3 in that it comprises several force sensors 123a-123c. As indicated by dashed lines in FIG. 4, the respective outputs from these force sensors 123a-123c are communicated to a controller 125 (e.g., comprising a processor and/or logic circuitry) for the suspension system 120. The controller 125 operates motors 122 that form part of the suspension system 120 so as to cause the position and/or orientation of the platform 111 to change, relative to the base 130, in response to forces sensed by the force sensors 123a-123c. In some examples, the controller 125 may operate the motors 122 to cause movements of the suspension system 120 that are expected to reduce (or minimize) the forces sensed by the force sensors 123a-123c. In such examples, the suspension system 120 may compliantly move in response to forces sensed by the force sensors 123a-123c.

It may be noted that, in the particular example shown in FIG. 4, the motors 122 are rotational motors that are integrated into the joints 121 of the suspension system 120; however, this is of course not essential and the motors 122 could be of any suitable type or have any suitable arrangement that can cause the position and/or orientation of the platform 111 to change, relative to the base 130, in response to forces sensed by the force sensors 123a-123c.

It may also be noted that force sensor 123a is integrated into coupling portion 112. It can therefore sense forces applied to the dental clamp 150 by the subject 10 during treatment. Accordingly, the output from force sensor 123a can be used, when the robotic dental system 1000′ is operated in a treatment mode, to control suspension system 120 to accommodate movement by the subject 10. In addition, or instead, the output from force sensor 123a can be used to detect undesirable or dangerous conditions, such as collisions between the treatment system 110 (and, in particular, the end effector 140 thereof) with the mouth or teeth of the subject 10 gross movements of the patient (e.g., when the patient sneezes). In response, the robotic dental system 1000′ can cause the dental clamp 150 to quickly release and/or can quickly remove the end effector 140 from the mouth of the subject 10, depending on the particular condition detected.

It may also be noted that force sensors 123b and 123c are integrated into, respectively, platform 111 and robotic arm 115. The output from one or both of such sensors can, for example, indicate that an operator is applying force to the treatment system 110. Hence, or otherwise, their output can be used when the robotic dental system 100 is operating in a compliant mode (which can, for instance, be a setup mode). More particularly, the output from one or both of the force sensors 123b and 123c of the treatment system 110 can be used to operate the robotic arm 115, so that an operator is able to reposition the robotic arm 115 in a desired arrangement, for example with the end effector 140 at a suitable location and/or orientation relative to a target tooth within the mouth of the subject 10. In such a compliant mode, the suspension system 120 continues to allow the subject 10 to alter their position and orientation, by enabling corresponding changes in the position and orientation of the platform 111, as described above.

A further (or alternative) refinement for assisting the operator of the robotic dental system 1000′ in suitably positioning the treatment system 110 is to provide, as part of the suspension system 120, a gross positioning system. Such a gross positioning system may be provided proximally of the joints 121 and linkages 124 shown in FIGS. 1-4. In other words, the gross positioning system connects the base 130 to the joints 121 and linkages 124. The gross positioning system may comprise a plurality of linkages connected by a plurality of joints (which may, in some examples, be passive joints, to simplify manufacture and/or to reduce cost). The gross positioning system would be used during a setup mode to bring the joints 121 and linkages 124 - and thereby the treatment system 110—into a desired position and/or orientation relative to the subject 10. The joints of the gross positioning system can then be locked so as to prevent relative movement of the gross positioning linkages during the treatment mode. The joints 121 and linkages 124 shown in FIGS. 1-4 are then able to move in the manner described above to allow the subject 10 to alter their position and orientation, by enabling corresponding changes in the position and orientation of the platform 111.

It should be noted that, although three force sensors 123a-123c and corresponding motors are shown and described with reference to FIG. 4, other embodiments can include other numbers of force sensors and/or corresponding motors.

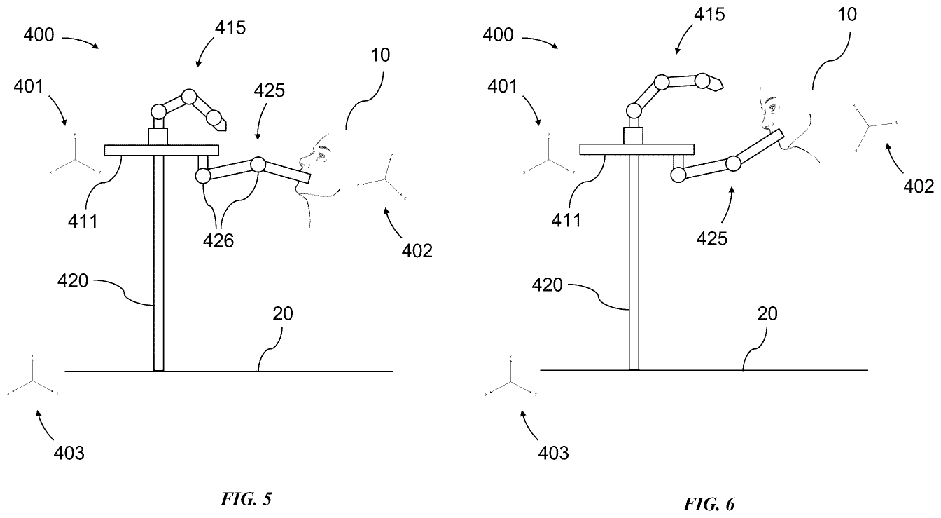

The advantages of the robotic dental systems of FIGS. 1-4 can be more fully appreciated by contrast with the comparative example of a robotic dental system illustrated in FIGS. 5 and 6. As shown, the system 400 of the comparative example includes a platform 411, which is supported above the floor 20 by a rigid support 420, and a robotic treatment arm 415, which is rigidly coupled to the platform 411 and which carries out a procedure on the subject 10. Notably, the system 400 of FIGS. 5 and 6 additionally includes a measurement arm 425, which is coupled to the subject 10, for example to the subject's teeth and/or jaw. The measurement arm 425 articulates freely (with the aid of articulated joints 426) when the subject 10 moves, and sensors within the measurement arm (not shown) determine the current position of the measurement arm. A controller translates the information from the sensors of the measurement arm 402 into compensatory movement of the treatment arm 415 to account for movements of the subject 10.

FIGS. 5 and 6 illustrate the frame of reference 401 of the platform 411 and the frame of reference 402 of the subject 10 at respective, different positions and orientations of the subject 10. Notice that the subject's frame of reference 402 is different between FIGS. 5 and 6, owing to the movement of the subject 10. However, the frame of reference 401 of the platform 411 is the same in both FIGS. 5 and 6. That is, the frame of reference 401 of the platform 411 does not change, in response to movement of the subject 10. Instead, the relationship of the frame of reference 401 of the platform 411 remains constant, relative to the frame of reference 403 of the floor/ground 20.

The system 400 of FIGS. 5 and 6 may generally be characterized as tracking movement of the subject 10 and actively compensating for such movement of the subject by controlling the movement of the robotic treatment arm 415 based on the measured movement of the subject 10. Such active compensation can introduce two errors into the subject tracking system. First, a measurement error occurs in the measurement arm 425. Second, a feedback delay occurs when processing the data and articulating the treatment arm 415, resulting in lag and additional errors as the subject moves.

Robotic systems implementing the design principles of the robotic treatment systems 1000, 1000′ described above with reference to FIGS. 1-4, such as the robotic dental systems described below with reference to FIGS. 8-16, can avoid the problems inherent in any system incorporating active feedback and correction and can avoid measurement errors and feedback delays by using a passive subject tracking system that does not require any active elements to react to and compensate for subject movement. In the robotic treatment systems 100 described above with reference to FIGS. 1-4 and other embodiments of the present disclosure, the subject can be rigidly attached to the system, preserving the spatial relationship relative to the base of the treatment arm, and the movable suspension system can carry the weight of the treatment such, including the platform 111 and all that is attached to thereto, such as the robotic arm 115, end effector 140, coupling portion 112, and dental clamp 150.

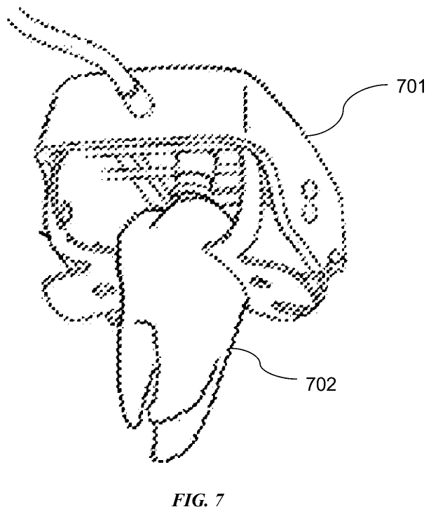

The robotic treatment systems 100 of FIGS. 1-4 and the embodiments of the present disclosure described below with reference to FIGS. 8-16 also differentiate from robotic dental systems that are so small that they sit on the subject's tooth 702, such as the system 701 shown in FIG. 7. Such a system (to the extent it could practically be implemented) would have no need of a suspension system, such as the suspension system 120 shown in FIGS. 1-3. Indeed, including a suspension system in such a robotic dental system would be antithetical to the fundamental design principle of the robotic dental system, which is to make the robotic dental system so lightweight that no support from, or engagement with, the ground is necessary. The examples of a robotic dental system 100 of FIGS. 1-4 and other embodiments of the present disclosure take a significantly different approach to such robotic dental systems that sit on a subject's tooth. Because the examples of a robotic dental system 100 of FIGS. 1-4 include a suspension system 120 that makes the treatment system 110 nearly weightless to the subject 10, it is possible for the treatment system 110 to include a sizable robotic arm 115, whose base is rigidly coupled to a platform 111 located outside the subject's mouth. Such an arrangement may afford the robotic arm 115 a large range of motion and/or the ability to treat multiple teeth without needing to be repositioned. In addition (or instead), such an arrangement can include larger (and therefore more powerful) motors and affords more flexibility over the types of end effector 140 that can be utilized by the robotic arm 115.

Attention is now directed to FIGS. 8-12, which show a robotic dental system 100 according to a first aspect of this disclosure. Like the robotic dental system 1000 shown in FIGS. 1-3, the robotic dental system 100 of FIGS. 8-13 comprises a treatment system 110 that includes a robotic arm 115, whose distal end 117 is configured to be coupled to an end effector 140, and whose proximal end 116 is coupled to a platform 111 of the treatment system 110. In addition, like the robotic dental system 1000 of FIGS. 1-3, the treatment system 110 of the robotic dental system 100 of FIGS. 8-13 is supported by a suspension system 120 that couples the platform 111 of the treatment system 110 to a base 130.

The suspension system 120 of the robotic dental system 100 of FIGS. 8-13 enables the treatment system 110 to accommodate movement by the subject 10 in generally the same manner as described above with reference to FIGS. 2-3, and accordingly includes a number of joints 121 and linkages 124 that articulate to permit movement of the platform 111, thereby accommodating movement by the subject 10. As will be discussed in more detail below, in the robotic dental system 100 of FIGS. 8-13, the joints 121 and linkages 124 of the suspension system 120 together provide the platform 111 with three degrees of translational freedom (e.g., translation in x, y and z directions) and three degrees of rotational freedom (e.g., rotation in roll, pitch and yaw directions).

Looking in more detail at FIG. 8, it may be noted that certain of the joints 121 of the suspension system 120 are indicated, with double-headed curved arrows, as rotating about respective vertical axes. These vertical axis revolute joints 121(a)(i)-(iii) are able to provide the platform 111 with various degrees of freedom.

In this regard, reference is directed to FIGS. 9 and 10, which are schematic top views of the robotic dental system 100 of FIG. 8 at respective points in time during movement of the platform as a result of articulation of the vertical axis revolute joints 121(a)(i)-(iii). As is apparent, the vertical axis revolute joints 121(a)(i)-(iii) permit the platform 111 to move within a horizontal plane. In the particular example shown in FIGS. 9 and 10, articulation of the first and second vertical axis revolute joints 121(a)(i), (ii) (and their connected linkages 124(i), 124(iii)) enables platform 111 to move to the right, as indicated by the arrow in FIG. 9. However, it will be appreciated that articulation of the first and second vertical axis revolute joints 121(a)(i), (ii) would equally enable the platform 111 to move upwards, downwards, or to the left (as seen from FIGS. 9 and 10) or a combination thereof.

It should further be noted that articulation of the third revolute joint 121(a)(iii) (which is beneath the platform 111 in FIGS. 9 and 10, and therefore not visible in FIGS. 9 and 10) enables the platform 111 to remain in the same orientation about a vertical axis passing through the platform 111, during the movement of the platform 111 to the right hand side of FIGS. 9 and 10. However, it will be understood that the third revolute joint 121(a)(iii) would equally be able to alter the orientation of the platform 111 about a vertical axis to a desired angular position, with suitable articulation of the third revolute joint 121(a)(iii). Hence, the combination of the three vertical axis revolute joints 121(a)(i)-(iii) is able to provide the platform 111 both with two degrees of translational freedom in a horizontal plane, and a rotational degree of freedom about a vertical axis.

It may further be seen from FIGS. 8-10 that, in the particular example shown, the proximal-most of the plurality of vertical axis revolute joints 121(a)(i) is located at the proximal end 1201 of the suspension system 120. Hence, or otherwise, the proximal-most of the plurality of vertical axis revolute joints 121(a)(i) may be the proximal-most joint in the suspension system 120. Such features may provide the suspension system 120 with a suitable amount of positioning flexibility, range and/or reach. However, it is by no means essential and, in other examples, a proximal-most vertical axis revolute joint could be disposed at some distance from the proximal end 1201 of the suspension system 120.

In some implementations, the vertical axis revolute joints 121(a)(i)-(iii) (or a subgroup of them) can be configured as active (i.e., powered) joints. Hence (or otherwise), one or more of the vertical axis revolute joints 121(a)(i)-(iii) may each be coupled to respective motors, each such motor being configured to cause movement of the corresponding one of the vertical axis revolute joints 121(a)(i)-(iii). Because the vertical axis revolute joints 121(a)(i)-(iii) are vertically oriented they will typically experience relatively little friction and/or stiction and hence relatively low powered and/or inexpensive motors can be utilised. Low powered motors can support patient safety, as they inherently limit the potential forces applied to the teeth of the subject 10 as a result of articulation of the active joints powered by such motors.

In certain examples, each motor may be integrated into the joint that it powers, though this is of course not essential. It will also be appreciated that such motors may be operated based on the output of force sensors (such as the force sensors 123a-123c described above with reference to FIG. 4) that are operable to sense (directly or indirectly) forces applied by the subject 10 to the dental clamp 150. Using the output of such force sensors, the vertical axis revolute joints 121(a)(i)-(iii) can, for example, be operated so as to minimize the force applied by the subject 10 to the dental clamp 150, thereby actively accommodating movement by the subject 10 during treatment.

It is however not essential that the vertical axis revolute joints 121(a)(i)-(iii) be active joints; in certain examples, all or some of them may be configured as passive joints. Because the vertical axis revolute joints 121(a)(i)-(iii) are vertically oriented they will typically experience relatively little friction and/or stiction. Hence (or otherwise), the vertical axis revolute joints 121(a)(i)-(iii) can be configured as passive joints with little impact on patient comfort, since the subject 10 does not need to provide significant force to the dental clamp 150 to cause passive vertical axis revolute joints 121(a)(i)-(iii) to articulate and accommodate their movement, during treatment.

Particularly (but not exclusively) where the suspension system 120 comprises vertical axis revolute joints 121(a)(i)-(iii), the robotic dental system 100′ may comprise a leveling system, which comprises moveable elements that are operable to maintain the suspension system 120 in a predetermined orientation with respect to gravity. This may reduce the friction and/or stiction experienced by the vertical axis revolute joints 121(a)(i)-(iii). Such a leveling system may, for example, be implemented as part of base 130 and could, in a specific example, comprise a plurality of feet whose position can be adjusted (e.g., automatically), to ensure that the robotic dental system 100′ is level. However, it is by no means essential that the leveling system may be implemented as part of base 130 and, in other examples, it could be implemented as part of the suspension system 120 itself. For example, one of the joints adjacent the proximal end 1201 of the suspension system 120 could be adjustable such that the remainder of the suspension system 120 (and thereby the suspension system 120 as a whole) is in a predetermined orientation with respect to gravity.

Returning to FIG. 8, it may be noted that, in the particular example of the robotic dental system 100 shown therein, the suspension system 120 comprises several elevation joints 121(b)(i)-(ii). These elevation joints 121(b)(i)-(ii) provide the platform 111 a translational degree of freedom in a vertical direction. In other words, they enable the platform 111 to move vertically upwards and downwards.

In the particular example shown in FIGS. 8-12, the elevation joints 121(b)(i)-(ii) are revolute joints, as indicated by the double-headed arrows in FIGS. 8, 11 and 12. Revolute joints may provide the suspension system 120 with suitable mechanical efficiency, which may support patient comfort, because the suspension system 120 responds quickly and with little resistance to movement by the subject 10.

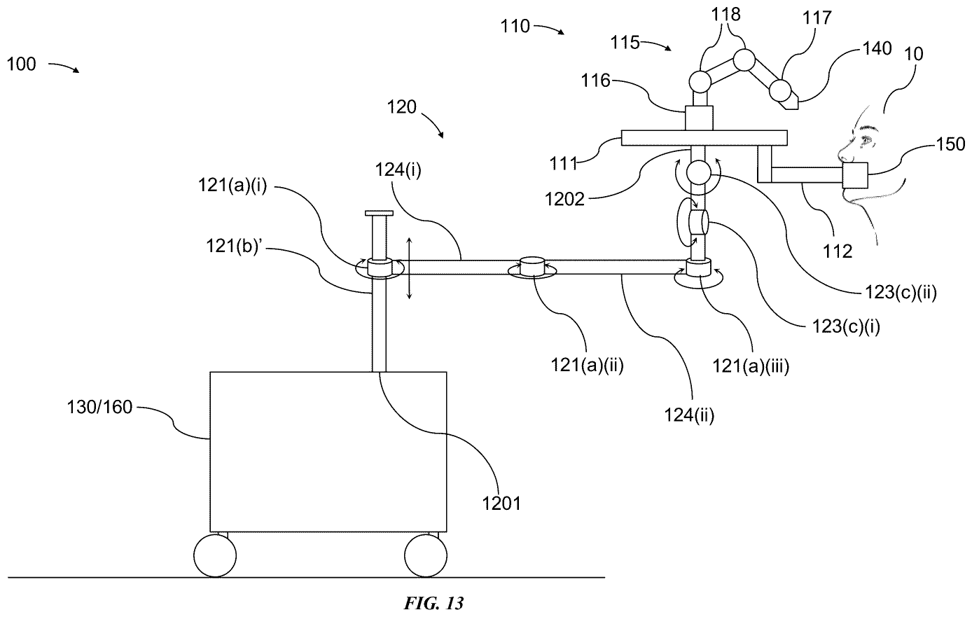

However, it is by no means essential that the elevation joints 121(b)(i)-(ii) are revolute joints and, in other examples, some or all of the elevation joints 121(b)(i)-(ii) could be prismatic or linear joints. In this regard, reference is directed to FIG. 13, which is a schematic side view of a further example of a robotic dental system 100 according to the same aspect of the disclosure as the example shown in FIGS. 8-12. The robotic dental system 100 is essentially the same as that shown in FIGS. 8-12, except that it comprises a prismatic elevation joint 121(b)′, which provides the platform 111 with a translational degree of freedom in a vertical direction.

Regardless of whether the elevation joints 212(b), 212(b)′ are revolute joints or prismatic, it is envisaged that, in various examples they may be configured as active joints, since they must counteract/work against the weight of the portion of the suspension system distal to them, and the weight of the treatment system 110. In the example shown in FIGS. 8-12, only a first elevation joint 121(b)(i) is configured as an active joint, for simplicity of manufacture and/or operation. However, this is of course not essential and, in other examples, other elevation joints 121(b)(i)-(ii), or all of the elevation joints 121(b)(i)-(ii), could be configured as active joints.

When configured as an active joint, each active elevation joint, such as the first elevation joint 121(b)(i) in FIGS. 8-12, is coupled to at least one motor, which is configured to cause movement of the active elevation joint in question. Such motor(s) may be controlled based on the output of force sensors (such as the force sensors 123a-123c described above with reference to FIG. 4) that are operable to sense (directly or indirectly) forces applied by the subject 10 to the dental clamp 150. Using the output of such force sensors, the first elevation joint 121(b)(i) (or any other active elevation joint) can, for example, be operated so as to minimize the force applied by the subject 10 to the dental clamp 150, thereby actively accommodating movement by the subject 10 during treatment.

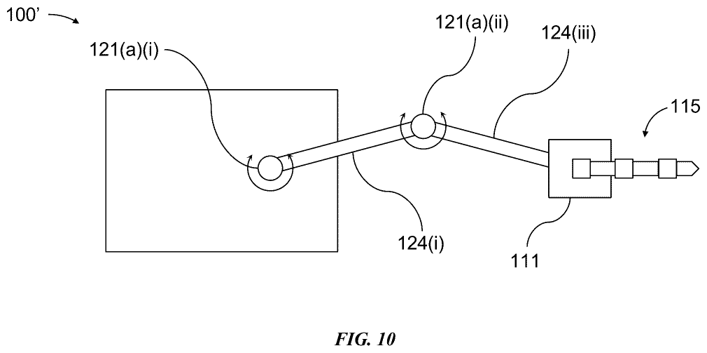

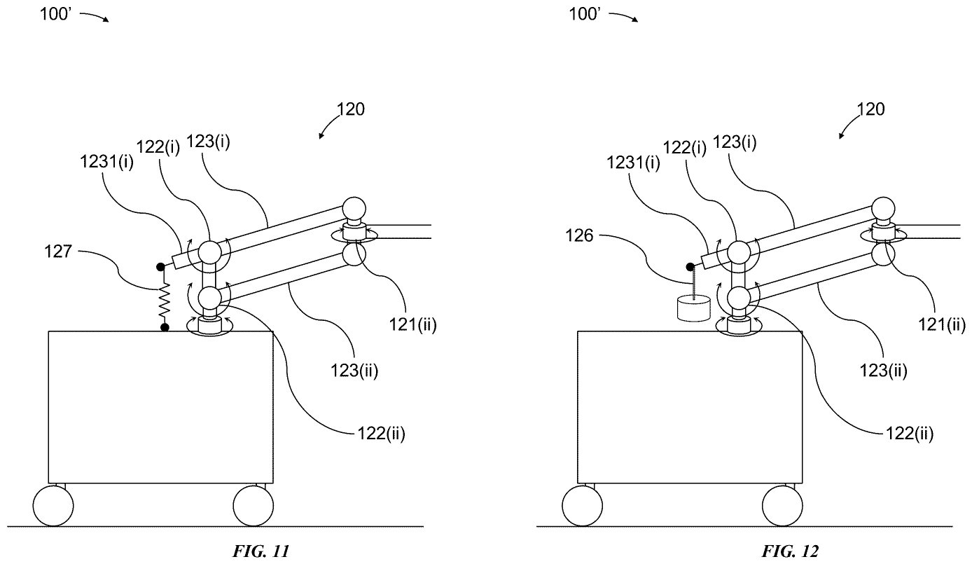

Furthermore, in some examples, each active elevation joint, such as the first elevation joint 121(b)(i) in FIGS. 8-12, may be coupled to one or more mechanical force-generating elements 126, 127, which applies force to counteract forces applied to the elevation joint in question by the portion of the suspension system distal to the elevation joint, as a result of the weight of that portion of the suspension system and the weight of the treatment system 110. Each of the mechanical force-generating elements 126, 127 can, for example, be a spring, a bungee cord, or a counterweight. In this regard, reference is directed to FIG. 11, which illustrates an example in which a spring 127 is attached to an extension 1231(i) of the linkage 123(i) connected to the first elevation joint 121(b)(i). FIG. 12 similarly shows an example in which a counterweight is attached to an extension 1231(i) of the linkage 123(i) connected to the first elevation joint 121(b)(i). As may be appreciated, in each case, the mechanical force-generating elements 126, 127 counteract the forces applied to the first elevation joint 121(b)(i) by the portion of the suspension system distal to the first elevation joint 121(b)(i). The use of such mechanical force-generating elements 126, 127 may therefore allow for relatively low-powered motors to be used to drive the active elevation joints. This can support patient safety, as the potential force/torque applied by the motors is inherently limited and thus the resulting force transferred through the suspension system 120 and applied to the teeth of the subject 10 is correspondingly limited if an unexpected event occurs.

In particular examples, the force (in the case of a prismatic joint) or torque (in the case of a revolute joint) that is applied to a given active elevation joint by the corresponding mechanical force-generating elements 126, 127 may, for instance, be greater than the maximum force or torque applied to that elevation joint by the corresponding motor(s). Indeed, it is envisaged that, in some implementations, the force or torque applied by the mechanical force-generating elements may be several times greater than the force or torque applied by the corresponding motors, e.g., 2, 4, 6, 8 or even 10 times greater.

Returning to FIG. 8, it may be noted that the suspension system 120 comprises two non-vertical axis rotational joints 123(c)(i)-(ii). In various examples, the non-vertical axis rotational joints 123(c)(i)-(ii) are active joints, given that they must counteract/work against the torque produced by the portion of the suspension system distal to them, and by the weight of the treatment system 110. Like elevation joints 121(b)(i)-(ii), where the non-vertical axis rotational joints 123(c)(i)-(ii) are active joints they are each coupled to at least one motor, which is configured to cause movement of the non-vertical axis rotational joints 123(c)(i)-(ii). Such motors may be controlled based on the output of force sensors (such as the force sensors 123a-123c described above with reference to FIG. 4) that are operable to sense (directly or indirectly) forces applied by the subject 10 to the dental clamp 150. Using the output of such force sensors, the non-vertical axis rotational joints 123(c)(i)-(ii) can, for example, be operated so as to minimize the force applied by the subject 10 to the dental clamp 150, thereby actively accommodating movement by the subject 10 during treatment.

Furthermore, active non-vertical axis rotational joints 123(c)(i)-(ii) can, in some examples, be coupled to one or more mechanical force-generating elements, such as springs, bungee cords, or other elastic elements, which apply torque that counteract torque applied to the non-vertical axis rotational joints 123(c)(i)-(ii) by the portion of the suspension system distal to the non-vertical axis rotational joints 123(c)(i)-(ii). This may allow for relatively low-powered motors to be used to drive the non-vertical axis rotational joints 123(c)(i)-(ii), which can support patient safety, as the potential torque applied by the motors is inherently limited and thus the resulting force transferred through the suspension system 120 and applied to the teeth of the subject 10 is correspondingly limited if an unexpected event occurs.

More generally, it is envisaged that the motors of all active joints in the robotic dental system 100 may be configured (through programming of their control systems, or through inherent physical limitations) such that the maximum force they can apply to the teeth of the subject 10 is less than 40 N, less than 20 N, less than 10 N, or even less than 10 N. Indeed, the motors of each active joint (or even each motor) may be configured to conform to such limits on the maximum force applied to the subject′ teeth.

While the particular example shown in FIG. 8 comprises two non-vertical axis rotational joints 123(c)(i)-(ii), it should be appreciated that this is not essential. In other examples, a single rotational joint that provides two rotational degrees of freedom about respective, non-vertical axes, could be utilised instead; for instance, a universal joint could be utilized.

Returning to FIG. 8, it may be noted that non-vertical axis rotational joints 123(c) (i)-(ii) are disposed at the distal end 1202 of the suspension system 120. Hence, or otherwise, they may be the distal-most joints within the suspension system 120. Consequently, the non-vertical axis rotational joints 123(c)(i)-(ii) are located distally of the elevation joints 121(b)(i)-(ii). Though not essential, such features may improve patient comfort, as less force needs to be applied by the patient to change the orientation of joints that are close to them.

It may also be seem from FIGS. 8-13 that, in contrast to the robotic dental system 1000 of FIGS. 1-3, the base 130 of the robotic dental system 100 of FIGS. 8-13 is not fixed to the ground, but rather is configured as a mobile cart 160 having a number of wheels 161 that permit an operator to move the robotic dental system 100 to a desired location. The mobile cart 160 may, in some examples, include various elements such as: a rechargeable power supply in electrical communication with an electric panel that provides charging ports for portable electronic devices; converters, transformers and surge protectors for a plurality of AC and DC receptacles that provide a power source for the equipment on-board the mobile cart 160, such as a user interface module and/or one or more computers storing application specific software for such a user interface module.

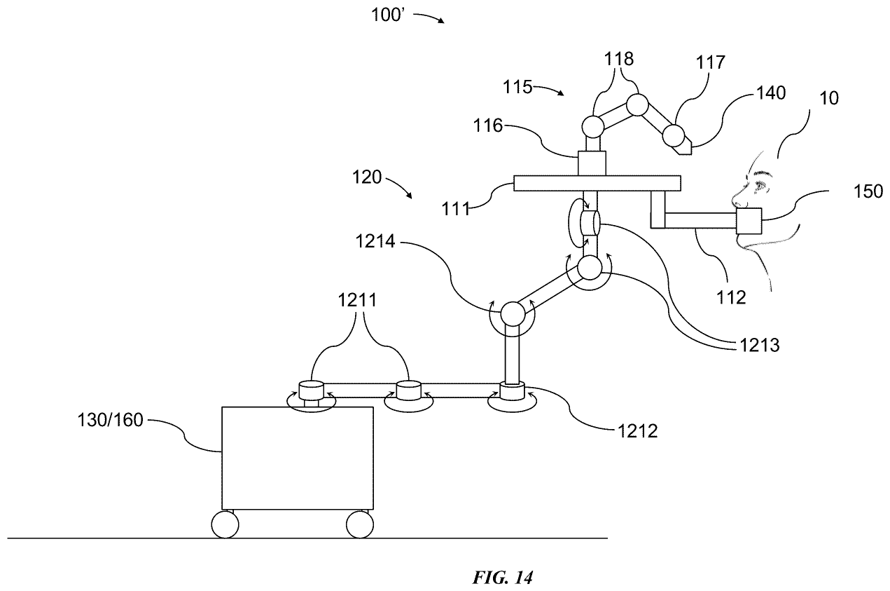

Reference is now direction to FIG. 14, which is a schematic side view of a further example of a robotic dental system 100′ according to the same aspect of this disclosure as the robotic dental systems described above with reference to FIGS. 8-13. The robotic dental system 100′ of FIG. 14 is substantially the same as the robotic dental system 100 described above with reference to FIGS. 8-13, with the exception of the suspension system 120. However, like in the suspension system 120 of the robotic dental system 100 of FIGS. 8-13, in the suspension system 120 of the robotic dental system 100 of FIG. 14, the joints 1211, 1212, 1213, 1215 and linkages 1241 of the suspension system 120 of FIG. 14 together provide the platform 111 with three degrees of translational freedom (e.g., translation in x, y and z directions) and three degrees of rotational freedom (e.g., rotation in roll, pitch and yaw directions).

In more detail, as shown in FIG. 14, the suspension system 120 of robotic dental system 100′ comprises an elevation joint 1214 that raises and lowers the platform 111, i.e., provides the platform 111 with a translational degree of freedom in the vertical direction. In the particular example shown, the elevation joint 1214 is an active joint. By contrast, a plurality of passive joints 1211 enable the platform 111 to move horizontally (i.e., in x and y directions). Put differently, these passive joints 1211 provide the platform 111 with two degrees of translational freedom in a horizontal plane. As may be seen, in the particular example shown in FIG. 14, the passive joints 1211 are rotational joints that permit the linkages to which they are connected to rotate about a vertical axis relative to one another. While such joints are by no means essential, they may be reliable and/or relatively compact.

Particularly (but not exclusively) where the robotic dental system 100′ comprises passive joints 1211 that enable the platform 111 to move horizontally, the robotic dental system 100′ may comprise a leveling system, which comprises moveable elements that are operable to maintain the suspension system 120 in a predetermined orientation with respect to gravity. For example, such a leveling system may be implemented as part of base 130 and could, in a specific example, comprise a plurality of feet whose position can be adjusted (e.g., automatically), to ensure that the robotic dental system 100′ is level. However, it is by no means essential that the leveling system may be implemented as part of base 130 and, in other examples, it could be implemented as part of the suspension system 120 itself. For example, one of the joints adjacent the base 130 could be adjustable such that the remainder of the suspension system 120 (and thereby the suspension system 120 as a whole) is in a predetermined orientation with respect to gravity.

It is considered that using active joints to provide the platform 111 with a translational degree of freedom in the vertical direction, but passive joints to provide the platform 111 with translational degrees of freedom in horizontal directions may provide a low cost and/or low manufacturing complexity robotic dental system 100′, particularly as compared with a system where all three translational degrees of freedom are provided by active joints.

It may also be noted that, in the particular example shown in FIG. 14, elevation joint 1214 is a rotational joint that enables the rotation of the linkages that it connects relative to one another about a horizontal axis. However, while this may be relatively cost effective, it is by no means essential and in other examples the elevation joint 1214 could, for example, be a linear or prismatic joint.

As further shown in FIG. 14, the suspension system 120 of the robotic dental system 100′ additionally comprises a passive joint 1212 that enables the platform 111 to rotate about a vertical axis, i.e. passive joint 1212 provides the platform 111 with a rotational degree of freedom in the yaw direction. As also shown, the suspension system 120 of the robotic dental system 100′ further comprises joints 1213 that enable the platform 111 to rotate about respective horizontal axes, i.e., they provide the platform 111 with rotational degrees of freedom in the pitch and roll directions. In contrast to joint 1212, joints 1213 are active joints - they are driven by motors. It is considered that using active joints to provide degrees of freedom in the pitch and roll directions (in which greater moments are typically exerted), but passive joint(s) to provide a degree of freedom in the yaw direction may provide a low cost and/or low manufacturing complexity robotic dental system 100′, particularly as compared with a system where all three rotational degrees of freedom are provided by active joints. It should be noted that, although degrees of freedom in the pitch and roll directions are provided by two active joints 1213, in other examples, rotation in the pitch and roll directions could be provided by a single active joint, such as an active ball joint (e.g., using spherical gear meshings), or more than two joints.

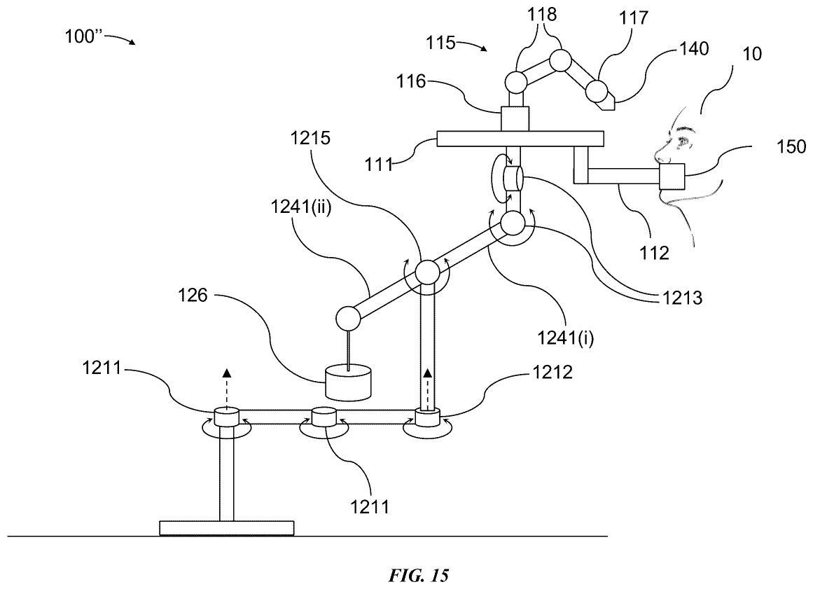

Reference is now direction to FIG. 15, which is a schematic side view of a still further example of a robotic dental system 100″ according to the same aspect of this disclosure as the described above with reference to FIGS. 8-14. The robotic dental system 100″ of FIG. 15 is essentially the same as the robotic dental system 100′ described above with reference to FIG. 14, except that its suspension system 120 includes a counterbalancing arrangement that counteracts the weight of the treatment system 110. More particularly, as shown, the suspension system 120 of robotic dental system 100″ comprises a passive elevation joint 1215, for raising and lowering platform 111, that is connected to a lever linkage 1241, between opposing first and second portions 1241(ii), 1241(ii) thereof. As may be appreciated, the first portion 1241(i) of the lever linkage 1241 supports the weight of treatment system 110. Conversely, the second portion 1241(ii) has mounted thereon a counterbalancing weight 126. This counterbalancing weight 126 substantially counterbalances the moment generated by the weight of treatment system 110. For example, the moment generated by the counterbalancing weight 126 about the elevation joint 1215 may be 70%, 80%, 90%, or more of the moment generated by the treatment system 110. This may result in little or no weight forces being applied to the teeth of subject 10.

Although in the counterbalanced arrangement described above the elevation joint 1215 is described as being a passive joint, it should be appreciated that it is by no means essential. Hence, in other examples, joint 1215 could be an active joint. In such examples, the counterbalancing arrangement could allow for a relatively small motor to be used within joint 1215, given that most of the weight of treatment system 110 would be counteracted by counterbalancing weight 126.

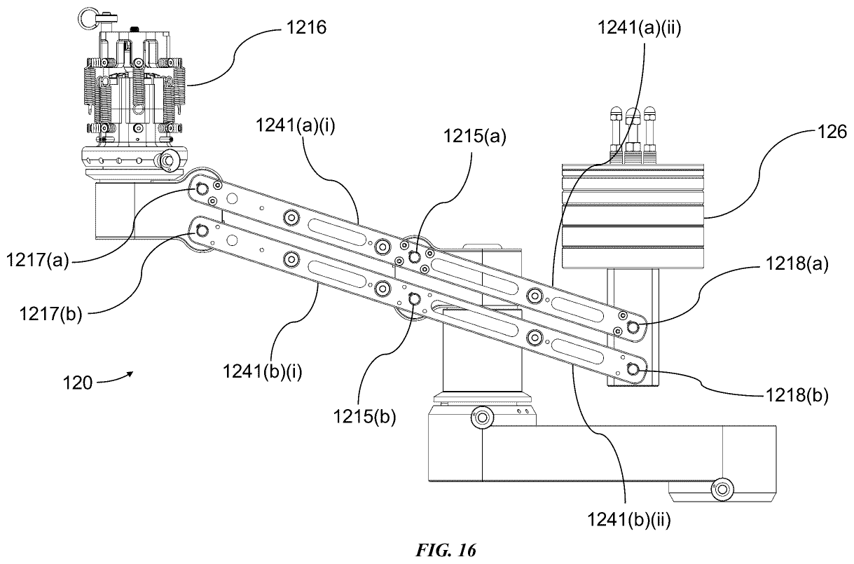

1214(b) Reference is now directed to FIG. 16, which shows a suspension system 120 with a more complex counterbalancing arrangement. In the suspension system 120 shown in FIG. 16, multiple lever linkages 1214(a), 1214(b) are provided, on whose respective second portions 1214(a)(ii), 1214(b)(ii) a counterbalancing weight 126 is mounted. Similarly to the arrangement shown in FIG. 15, this counterbalancing weight 126 counteracts the moment generated by the treatment system 110 and applied to the respective first portions 1214(a)(i), 1214(a)(ii) of the lever linkages 1214(a), 1214(b). As may be appreciated from FIG. 16, the lever linkages 1214(a), 1214(b) are connected to respective joints 1215(a), 1215(b) that enable the lever linkages 1214(a), 1214(b) to rotate about parallel, vertically-offset horizontal axes. Similarly, the first portions 1214(a)(i), 1214(a)(ii) of the lever linkages 1214(a), 1214(b) are connected at parallel, vertically-offset joints 1217(a), 1217(b) to a treatment system connecting assembly 1216 and the second portions 1214(a)(ii), 1214(b)(ii) are connected at parallel, vertically-offset joints 1218(a), 1218(b) to the counterbalancing weight 126. This parallel motion arrangement maintains the orientation of the treatment system connecting assembly 1216 and the counterbalancing weight 126 with respect to gravity. The treatment system connecting assembly 1216 connects to platform 111 and acts as a ball and socket joint, providing the platform 111 with rotational degrees of freedom in the pitch and roll directions. In the particular example shown, the treatment system connecting assembly 1216 includes a number of springs (or other biasing members) that bias the platform 111 towards a horizontal orientation (or, in other examples, some other specific orientation with respect to gravity).

It should be appreciated that the robotic dental systems described herein may carry out various dental procedures. Because of their high level of accuracy, it is envisaged that the robotic dental systems described herein are particularly (but by no means exclusively) suitable for dental procedures that are carried out on the teeth themselves, as opposed to procedures carried out on, for example, the jawbone, where less accuracy is typically needed. In a specific example, the robotic dental systems may be configured (e.g. by suitable programming of processor(s) 181 and/or by storage of suitable instructions on computer-readable storage medium 182 and/or by the provision of a suitable end effector 140, such as a dental drill) so as to carry out tooth preparation in advance of the installation of a dental prosthetic, such as a crown or bridge. In another specific example, the dental systems may be configured (e.g., by suitable programming of processor(s) 181 and/or by storage of suitable instructions on computer-readable storage medium 182 and/or by the provision of a suitable end effector 140, such as a dental drill) to carry out removal of carious lesions of teeth.

Furthermore, while the above examples of robotic dental systems include only one robotic arm, it is envisaged that, in further examples two (or potentially more) robotic arms could be provided as part of the treatment system 110, rigidly coupled to the platform 111. In such examples, each robotic arm could be provided with a different end effector 140. In addition, or instead, the robotic dental system 100 could be configured (e.g., by suitable programming of the at least one processor 181 of the control system 180) such that the robotic arms (or a group of them) operate on a target tooth simultaneously or sequentially.

Still further, although in the above examples the dental clamp 150 is described as directly contacting and engaging with the one or more teeth of the subject 10, it is envisaged that, in other examples, the dental clamp 150 could additionally clamp onto other parts of the mouth of the subject 10 and/or could additionally clamp onto the jaw of the subject 10. Furthermore, in aspects of this disclosure that are different and/or broader than those exemplified above, the dental clamp could clamp onto the jaw of the subject 10 instead of the teeth of the subject 10. In still broader aspects, it is envisaged that a robotic surgical system or a robotic diagnostic system or a robotic treatment system could be provided that, respectively, operates on, diagnoses conditions in, or treats, a part of the body other than the teeth and that clamps onto that body part or an adjacent one, but makes use of a platform and suspension system substantially similar to those described above. In such robotic systems, the treatment system might not comprise a robotic arm. For instance, in a robotic diagnostic system, the treatment system might comprise an imaging device, such as an x-ray imaging device, that is coupled to the platform by a means other than a robotic arm, for example using a mount that enables the imaging device to be repositioned and/or reoriented with respect to the platform.

### Definitions

As used herein, the following terms shall have the following meanings, unless context indicates otherwise.

“Pressure” means a force applied perpendicular to a surface of an object per unit area over which the force is distributed. A non-zero pressure that is less than an ambient pressure, or less than a pressure in a reference location such as a suction material input port, is referred to as a “partial vacuum,” but is nonetheless considered to be a pressure. Partial vacuum is measured in units of pressure, typically as a subtraction relative to ambient atmospheric pressure on Earth or the pressure in the reference location. “Gauge pressure” is pressure relative to an ambient, usually atmospheric, pressure, and a negative gauge pressure indicates a partial vacuum.

“Continually” means continuously or repeatedly, although not necessarily in perpetuity. The term continually encompasses periodically and occasionally. Continually generating a signal means generating a continuously varying signal over time or generating a series of (more than one) discrete signals over time. Continually generating a value, such as an error value, means generating a continuously varying value, such as an analog value represented by a continuously varying voltage, or generating a series of (more than one) discrete values over time, such as a series of digital or analog values.

While the present disclosure is described through the above-described exemplary embodiments, modifications to, and variations of, the illustrated embodiments may be made without departing from the concepts disclosed herein. For example, although specific parameter values, such as materials and dimensions, may be recited in relation to disclosed embodiments, within the scope of the invention, the values of all parameters may vary over wide ranges to suit different applications. Unless otherwise indicated in context or would be understood by one of ordinary skill in the art, terms such as “about” mean within ±20%.

As used herein, including in the claims, the term “and/or,” used in connection with a list of items, means one or more of the items in the list, i.e., at least one of the items in the list, but not necessarily all the items in the list. As used herein, including in the claims, the term “or,” used in connection with a list of items, means one or more of the items in the list, i.e., at least one of the items in the list, but not necessarily all the items in the list. “Or” does not mean “exclusive or.”

As used herein, including in the claims, an element described as being configured to perform an operation “or” another operation is met by an element that is configured to perform only one of the two operations. That is, the element need not be configured to operate in one mode in which the element performs one of the operations, and in another mode in which the element performs the other operation. The element may, however, but need not, be configured to perform more than one of the operations.

Although aspects of embodiments may be described with reference to flowcharts and/or block diagrams, functions, operations, decisions, etc. of all or a portion of each block, or a combination of blocks, may be combined, separated into separate operations or performed in other orders. References to a “module,” “operation,” “step” and similar terms are for convenience and not intended to limit their implementation. All or a portion of each block, module, operation, step or combination thereof may be implemented as computer program instructions (such as software), hardware (such as combinatorial logic, Application Specific Integrated Circuits (ASICs), Field-Programmable Gate Arrays (FPGAs), processor or other hardware), firmware or combinations thereof.

The controller, etc. or portions thereof may be implemented by one or more suitable processors executing, or controlled by, instructions stored in a memory. Each processor may be a general-purpose processor, such as a central processing unit (CPU), a graphic processing unit (GPU), digital signal processor (DSP), a special purpose processor, etc., as appropriate, or combination thereof.

The memory may be random access memory (RAM), read-only memory (ROM), non-volatile memory (NVM), non-volatile random-access memory (NVRAM), flash memory or any other memory, or combination thereof, suitable for storing control software or other instructions and data. Instructions defining the functions of the present invention may be delivered to a processor in many forms, including, but not limited to, information permanently stored on tangible non-transitory non-writable storage media (e.g., read-only memory devices within a computer, such as ROM, or devices readable by a computer I/O attachment, such as CD-ROM or DVD disks), information alterably stored on tangible non-transitory writable storage media (e.g., floppy disks, removable flash memory and hard drives) or information conveyed to a computer through a communication medium, including wired or wireless computer networks. Moreover, while embodiments may be described in connection with various illustrative data structures, database schemas and the like, systems may be embodied using a variety of data structures, schemas, etc.

Disclosed aspects, or portions thereof, may be combined in ways not listed herein and/or not explicitly claimed. In addition, embodiments disclosed herein may be suitably practiced, absent any element that is not specifically disclosed herein. Accordingly, the invention should not be viewed as being limited to the disclosed embodiments.

As used herein, numerical terms, such as “first,” “second” and “third,” are used to distinguish respective robot arm links, joints, etc. from one another and are not intended to indicate any particular order or total number of links or joints in any particular embodiment. Thus, for example, a given embodiment may include only a second link and a third joint.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the present disclosure described herein may be employed in practicing the present disclosure. It is intended that the following claims define the scope of the invention and that methods and structures within the scope of these claims and their equivalents be covered thereby.

What is claimed is:

1. A robotic dental system, comprising:
a treatment system, which comprises:
a robotic arm, a distal end of which is configured to be coupled to an end effector; and
a platform, to which a proximal end of the robotic arm is coupled, the platform comprising a coupling portion for rigidly coupling to a dental clamp, which is configured to be rigidly clamped to one or more teeth of a subject, the platform and coupling portion being configured such that, when the dental clamp is rigidly clamped to the one or more teeth and the dental clamp is rigidly coupled to the coupling portion, a position and orientation of the platform remain fixed, relative to the one or more teeth ;
a base; and
a suspension system, comprising a proximal end, which is coupled to the base, and a distal end, which is coupled to the platform, wherein the suspension system supports a weight of the treatment system, and is configured such that, when the robotic dental system is operating in a treatment mode, with the dental clamp rigidly clamped to the one or more teeth and the dental clamp rigidly coupled to the coupling portion, the suspension system permits a the position and orientation of the platform to change, relative to the base, in response to forces applied by the one or more teeth to the dental clamp, thereby accommodating changes in a the position, orientation, and both of the one or more teeth by enabling corresponding changes in the position, orientation, or both of the platform,
wherein the suspension system comprises a plurality of linkages, connected by a plurality of joints, the plurality of linkages and the plurality of joints together providing the platform with three degrees of translational freedom and three degrees of rotational freedom.

2. The system of claim 1, wherein the plurality of joints comprises a plurality of vertical axis revolute joints, each of which is connected to one or more of the plurality of linkages, and enables said one or more linkages to rotate about a vertical axis corresponding to the revolute joint in question, and
wherein the plurality of vertical axis revolute joints provides the platform with two degrees of translational freedom in a horizontal plane.

3. The system of claim 2, wherein the plurality of vertical axis revolute joints comprises three vertical axis revolute joints, and wherein the plurality of vertical axis revolute joints additionally provides the platform with a rotational degree of freedom about a vertical axis.

4. The system of claim 1, wherein a proximal-most of the plurality of vertical axis revolute joints is located at the proximal end of the suspension system.

5. The system of claim 1, wherein one or more of the plurality of vertical axis revolute joints are coupled to respective motors, each of which is configured to cause movement of the corresponding vertical axis revolute joint.

6. The system of claim 1, wherein the plurality of joints comprise one or more elevation joints, which provide the platform at least with a translational degree of freedom in a vertical direction, and wherein at least a first elevation joint of the one or more elevation joints is coupled to at least one motor, which is configured to cause movement of the first elevation joint.

7. The system of claim 6, wherein the first elevation joint is coupled to at least one mechanical force-generating element, which applies force that counteracts force applied to the elevation joint by a portion of the suspension system distal to the elevation joint.

8. The system of claim 7, wherein the one or more elevation joints are revolute joints.

9. The system of claim 8, wherein a maximum torque applied to the elevation joint by the at least one mechanical force-generating element is greater than a maximum torque applied to the elevation joint by the at least one motor.

10. The system of claim 6, wherein the elevation joint is a prismatic joint.

11. The system of claim 10, wherein a maximum force applied to the elevation joint by the at least one mechanical force-generating element is greater than a maximum force applied to the elevation joint by the at least one motor.

12. The system of claim 1, wherein the plurality of joints comprises one or more non-vertical axis rotational joints, which collectively provide the platform with two rotational degrees of freedom about respective, non-vertical axes.

13. The system of claim 12, wherein the plurality of joints comprise one or more elevation joints, which provide the platform at least with a translational degree of freedom in a vertical direction, and wherein at least a first elevation joint of the one or more elevation joints is coupled to at least one motor, which is configured to cause movement of the first elevation joint, and wherein the one or more non-vertical axis rotational joints are located distally of the elevation joint.

14. The system of claim 12, wherein the plurality of joints comprises a plurality of vertical axis revolute joints, each of which is connected to one or more of the plurality of linkages, and enables said one or more linkages to rotate about a vertical axis corresponding to the revolute joint in question,
wherein the plurality of vertical axis revolute joints provides the platform with two degrees of translational freedom in a horizontal plane, and
wherein the one or more non-vertical axis rotational joints are located distally of the plurality of vertical axis revolute joints.

15. The system of claim 12, wherein the one or more non-vertical axis rotational joints are located at the distal end of the suspension system.

16. The system of claim 12, wherein the one or more non-vertical axis rotational joints coupled to one or more motors, configured to cause movement of the one or more non-vertical axis rotational joints.

17. The system of claim 16, wherein the one or more non-vertical axis rotational joints are coupled to one or more mechanical force-generating elements, which apply torque that counteract torque applied to the one or more non-vertical axis rotational joints by a portion of the suspension system distal to the one or more non-vertical axis rotational joints.

18. The system of claim 17, wherein a maximum torque applied to the one or more non-vertical axis rotational joints by the one or more mechanical force-generating elements is greater than a maximum torque applied to the elevation joint by the one or more motors.
